(19) United States
(12) Patent Application Publication
(10) Pub. No.: US 2001/0018609 A1
(43) Pub. Date: Aug. 30, 2001

(54) SEAMLESS BRAIDED OR SPUN STENT COVER

(76) Inventor: SCOTT SMITH, CHASKA, MN (US)

Correspondence Address: HOFFMANN & BARON, 6900 JERICHO TURNPIKE, SYOSSET, NY, 11791

(21) Appl. No.: 09/371,171

(22) Filed: Aug. 11, 1999

(51) Int. Cl. A61F 2/06

(52) U.S. Cl. 623/1.13; 623/1.51; 623/1.44

(57) ABSTRACT

A composite stent-graft tubular prosthesis includes a non-continuous tubular body formed of polytetrafluoroethylene components, providing axial and circumferential compliance to said prosthesis and a circumferentially distensible stent.

### FIELD OF THE INVENTION

The present invention relates generally to a tubular implantable prosthesis formed of porous expanded polytetrafluoroethylene. More particularly, the present invention relates to a composite, multi-layered endoprosthesis having increased axial and radial compliance.

### BACKGROUND OF THE RELATED TECHNOLOGY

An intraluminal prosthesis is a medical device commonly known to be used in the treatment of diseased blood vessels. An intraluminal prosthesis is typically used to repair, replace, or otherwise correct a damaged blood vessel. An artery or vein may be diseased in a variety of different ways. The prosthesis may therefore be used to prevent or treat a wide variety of defects such as stenosis of the vessel, thrombosis, occlusion, or an aneurysm.

One type of endoluminal prosthesis used in the repair of diseases in various body vessels is a stent. A stent is a generally longitudinal tubular device formed of biocompatible material which is useful to open and support various lumens in the body. For example, stents may used in the vascular system, urogenital tract and bile duct, as well as in a variety of other applications in the body. Endovascular stents have become widely used for the treatment of stenosis, strictures, and aneurysms in various blood vessels. These devices are implanted within the vessel to open and/or reinforce collapsing or partially occluded sections of the vessel.

Stents are generally open ended and are radially expandable between a generally unexpended insertion diameter and an expanded implantation diameter which is greater than the unexpended insertion diameter. Stents are often flexible in configuration, which allows them to be inserted through and conform to tortuous pathways in the blood vessel. The stent is generally inserted in a radially compressed state and expanded either through a self-expanding mechanism, or through the use of balloon catheters.

A graft is another type of commonly known type of intraluminal prosthesis which is used to repair and replace various body vessels. A graft provides an artificial lumen through which blood may flow. Grafts are tubular devices which may be formed of a variety of material, including textiles, and non-textile materials. One type of non-textile material particularly useful as an implantable intraluminal prosthesis is polytetrafluoroethylene (PTFE). PTFE exhibits superior biocompatability and low thrombogenicity, which makes it particularly useful as vascular graft material in the repair or replacement of blood vessels. In vascular applications, the grafts are manufactured from expanded polytetrafluoroethylene (ePTFE) tubes. These tubes have a microporous structure which allows natural tissue ingrowth and cell endothelization once implanted in the vascular system. This contributes to long term healing and patency of the graft. These tubes may be formed from extruded tubes or may be formed from a sheet of films formed into tubes.

Grafts formed of ePTFE have a fibrous state which is defined by interspaced nodes interconnected by elongated fibrils. The spaces between the node surfaces that is spanned by the fibrils is defined as the internodal distance (IND). Porosity of a graft is measured generally by IND. In order of proper tissue ingrowth and cell endothelization, grafts must have sufficient porosity obtained through expansion. When the term expanded is used to describe PTFE, it is intended to describe PTFE which has been stretched, in accordance with techniques which increase IND and concomitantly porosity. The stretching may be in uni-axially, bi-axially, or multi-axially. The nodes are spaced apart by the stretched fibrils in the direction of the expansion. Properties such as tensile strength, tear strength and radial (hoop) strength are all dependent on the expansion process. Expanding the film by stretching it in two directions that are substantially perpendicular to each other, for example longitudinally and transversely, creates a biaxially oriented material. Films having multi-axially-oriented fibrils may also be made by expanding the film in more than two directions. Porous ePTFE grafts have their greatest strength in directions parallel to the orientation of their fibrils. With the increased strength, however, often comes reduced flexibility.

While ePTFE has been described above as having desirable biocompatability qualities, tubes comprised of ePTFE, as well as films made into tubes, tend to exhibit axial stiffness, and minimal radial compliance. Longitudinal compliance is of particular importance to intraluminal prosthesis as the device must be delivered through tortuous pathways of a blood vessel to the implantation site where it is expanded. A reduction in axial and radial flexibility makes intraluminal delivery more difficult.

Composite intraluminal prosthesis are known in the art. In particular, it is known to combine a stent and a graft to form a composite medical device. Such composite medical devices provide additional support for blood flow through weakened sections of a blood vessel. In endovascular applications the use of a composite graft or a stent/graft combination is becoming increasingly important because the combination not only effectively allows the passage of blood therethrough, but also ensures patency of the implant. Where ePTFE is used as a graft component, the ePTFE is typically applied as a sheet or tube about the inner surface, outer surface, or both surfaces of the stent. Depending upon the specific properties of the ePTFE employed, various properties of the composite will be affected. For example, the ePTFE may affect the porosity and permeability of the composite. Also, the ePTFE will result in reduction of the mechanical compliance of the stent. So while composite prosthesis, especially those consisting of ePTFE, while exhibiting superior biocompatability qualities, they may also exhibit a decrease in other properties such as axial and radial compliance. It is therefore desirable to provide an ePTFE composite intraluminal prosthesis which exhibits increased performance characters such as axial and radial compliance.

### SUMMARY OF THE INVENTION

The present invention comprises a composite ePTFE vascular prosthesis. The composite has two layers; a discontinuous tubular ePTFE layer, and a circumferentially distensible support structure.

One advantage of the present invention is that it provides an improved composite ePTFE intraluminal prosthesis exhibiting increased axial and radial compliance.

Another advantage of the present invention is that it provides an improved composite ePTFE intraluminal prosthesis exhibiting increased axial and radial compliance, flexibility, and greater tissue ingrowth, through the use of multiaxial fibril direction in a non-continuous outer ePTFE tubular body.

In a desired embodiment, the present invention provides a three layer composite intraluminal prosthesis for implantation which may have a substantially continuous ePTFE tubular body, in combination with a non-continuous outer ePTFE tubular body formed by tubularly assembled polytetrafluoroethylene strips, or components, and a circumferentially distensible support structure between the two PTFE layers, with the PTFE layers secured together by, or through, the distensible support structure. The components or strips comprising the non-continuous tubular body possess a longitudinal length and a width, with said longitudinal length being greater than said width. The non continuous, tubular assembled strips providing axial and circumferential compliance to said prosthesis.

It is yet another advantage of the present invention to provide an improved method of forming such composites by spirally wound strips of PTFE. One method of forming an intraluminal prosthesis stent/graft with axial and circumferential compliance is provided by spirally wrapping strips of the non-continuous PTFE tubular outer body over a mandrel to form the non-continuous tubular layer, and attaching the support structure atop the tubular layer. Alternatively, the PTFE strips may be wound atop the support structure. Another PTFE layer, either a continuous tubular layer or a longitudinally non-continuous layer may be assembled over, or under, respectively, the distensible support structure.

### BRIEF DESCRIPTION OF THE DRAWINGS

FIG. 1 is a plane view of a non-continuous tubular layer of opposed, spirally wound PTFE components, which may form the inner or outer tubular layer of the composite prosthesis of the present invention.

FIG. 2 is a plane view of another embodiment of the non-continuous PTFE layer of the composite prosthesis of the present invention, illustrating interwoven, opposed, spirally wound PTFE components atop the support structure of the composite prosthesis according to the present invention.

FIG. 3 is a plane view of spirally wound layers of PTFE components, forming the longitudinally non-continuous layer of the composite prosthesis of the present invention, including a third pass winding, and illustrating a segmented mandrel, for forming the composite stent graft prosthesis according to the present invention.

FIG. 4 shows a perspective view of the wound or interwoven non-continuous tubular body of another embodiment of the present invention, with a support structure and continuous tubular inner body.

FIG. 5 shows an enlarged perspective view of the exterior surface of one embodiment of the PTFE components of the present invention, showing woven PTFE tapes.

FIG. 6 shows an enlarged perspective of the exterior surface of another embodiment of PTFE components of the present invention, illustrating interwoven threads of braided PTFE filaments.

### DETAILED DESCRIPTION OF THE INVENTION

The prosthesis of the preferred embodiment of the present invention is a composite implantable intraluminal prosthesis which is particularly suited for use as a vascular graft. The composite prosthesis of the present invention includes a graft structure with circumferentially distensible support structure and a noncontinuous layer of wound PTFE components. Desirably, the composite may also include a continuous ePTFE layer, with the circumferentially distensible support structure interposed between these PTFE layers. The present description is meant to describe the preferred embodiments, and is not meant to limit the invention in any way.

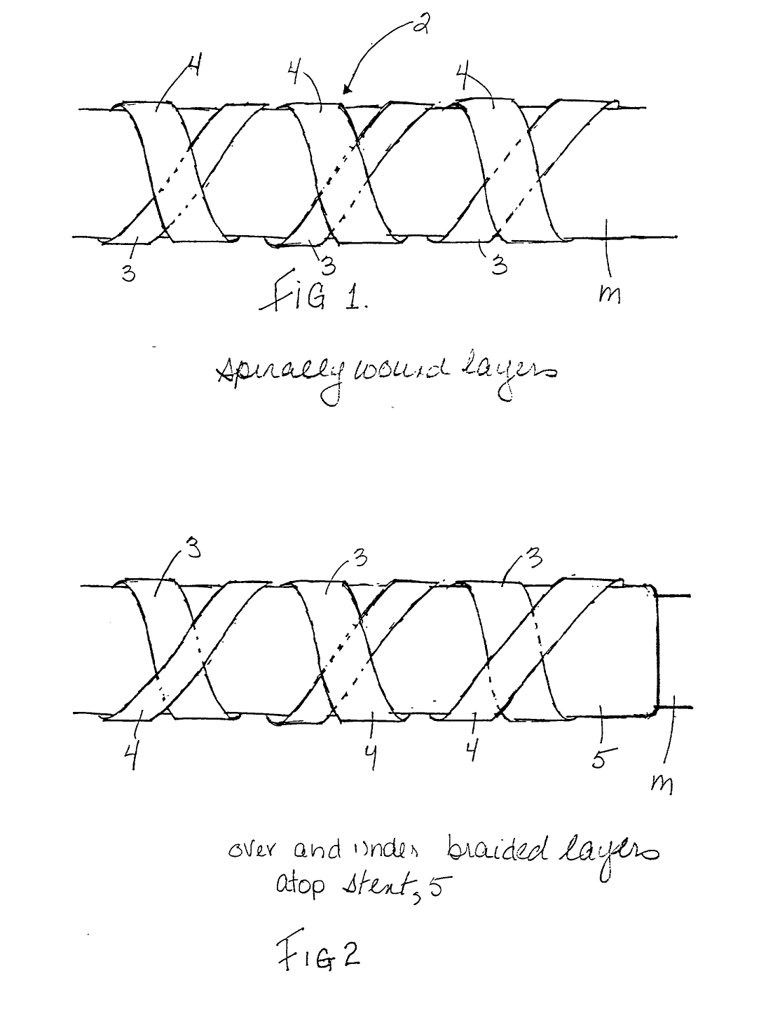

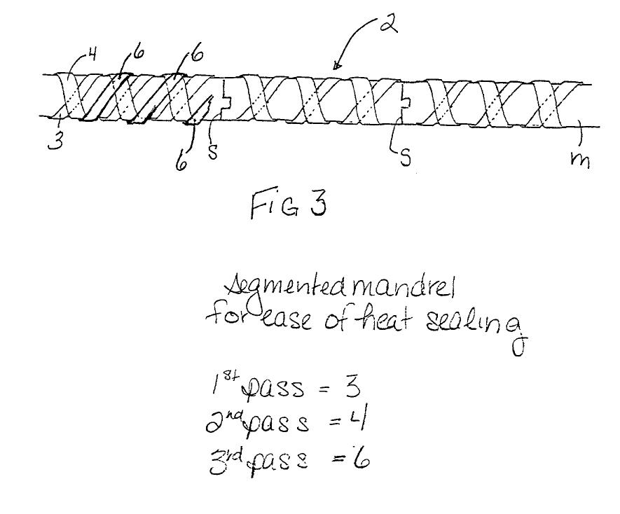

Shown in FIG. 1 is a longitudinally discontinuous tubular PTFE body, shown generally at 2, which forms one of the layers of the composite. The tubular body is formed by wrapping at least two PTFE components, such as strips 3, 4, in opposed spirals, about a distensible tubular support structure shown generally at 5, or directly around a mandrel m, to form a tubular body 2 without a seam. The tubular body may consist of any number of PTFE components spirally wound around the mandrel, to form a longitudinally non-continuous tubular body. When desired, the non-continuous layer may be wound about the support structure as shown in FIG. 2. Alternatively, the support structure may be used as a mandrel, for forming the non-continuous PTFE tubular body.

Continuous, as used herein, refers to a tubular structure whose surface extends substantially uninterrupted throughout the longitudinal length thereof. In the case of an extruded tube, the tubular structure is completely uninterrupted. In the case of a sheet formed tube there are no transverse interruptions. As is known in the art, a substantially uninterrupted tubular PTFE structure exhibits enhanced strength and sealing properties when used as a vascular graft, but little radial or axial compliance.

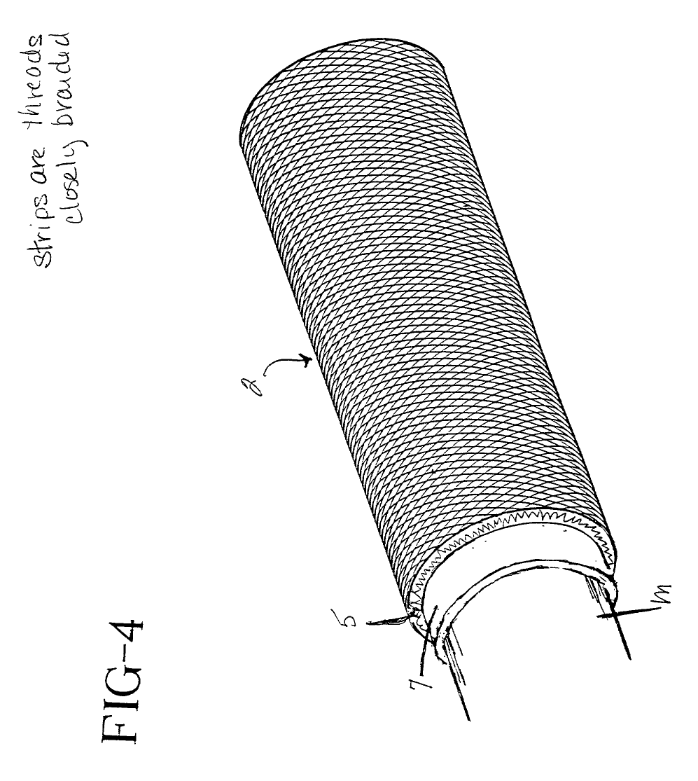

FIG. 2 depicts a tubular body where strips 3 and 4 are interwoven through each other according to the present invention atop the circumferentially distensible support structure, or stent 5. Distensible, as used herein, refers to a stent which may be expanded and contracted radially. The stent, 5, may be fastened to the non-continuous tubular body, or simply assembled therewith to form a composite structure, with a stent side and a PTFE side. A three layer composite prosthesis may be made by (pre)adding a continuous ePTFE tubular body, as shown at 7 in FIG. 4. Alternatively, the non-continuous layer may be formed on the mandrel (i.e. FIGS. 1 or 3), the support structure placed thereon, and a non-continuous or a continuous PTFE layer placed atop the support structure. In constructing the longitudinally non-continuous tubular body of the present invention it is not necessary for the components to be of similar width, or wound with the same number of turns per inch, or in the same direction.

Various stent types and stent constructions may be employed in the invention. Among the various stents useful include, without limitation, self-expanding stents and balloon expandable stents. The stents may be capable of radially contracting, as well, and in this sense can best be described as radially distensible or deformable. Self-expanding stents include those that have a spring-like action which causes the stent to radially expand, or stents which expand due to the memory properties of the stent material for a particular configuration at a certain temperature. Nitinol is one material which has the ability to perform well while both in spring-like mode, as well as in a memory mode based on temperature. Other materials are of course contemplated, such as stainless steel, platinum, gold, titanium and other bicompatible metals, as well as polymeric stents.

The configuration of the stent may also be chosen from a host of geometries. For example, wire stents can be fastened into a continuous helical pattern, with or without a wave-like or zig-zag in the wire, to form a radially deformable stent. Individual rings or circular members can be linked together such as by struts, sutures, welding or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like.

The circumferentially tubular distensible support structure, or stent, may be formed of an elongate wire, helically wound, which may be compacted with the non-continuous tubular body to form a radially expandable stent composite. The stent may be of the type described in U.S. Pat. No. 5,575,816 to Rudnick, et al. The distensible support structure, or stent, may be either of the balloon-expanded or self-expanded type. Stents of this type are typically introduced intraluminally into the body, and expanded at the implantation site.

FIG. 3 shows an alternate assembly wherein the PTFE components 3, 4, and 6 are assembled on the mandrel in multiple passes. As shown, the last pass winds component 6 over the opposed winding of components 3 and 4. Any number of components may be used to form the non-continuous tubular bodies. The windings are made helically, in any direction, along the mandrel or support structure. The mandrel may be constructed of segments for ease of heat sealing the composites.

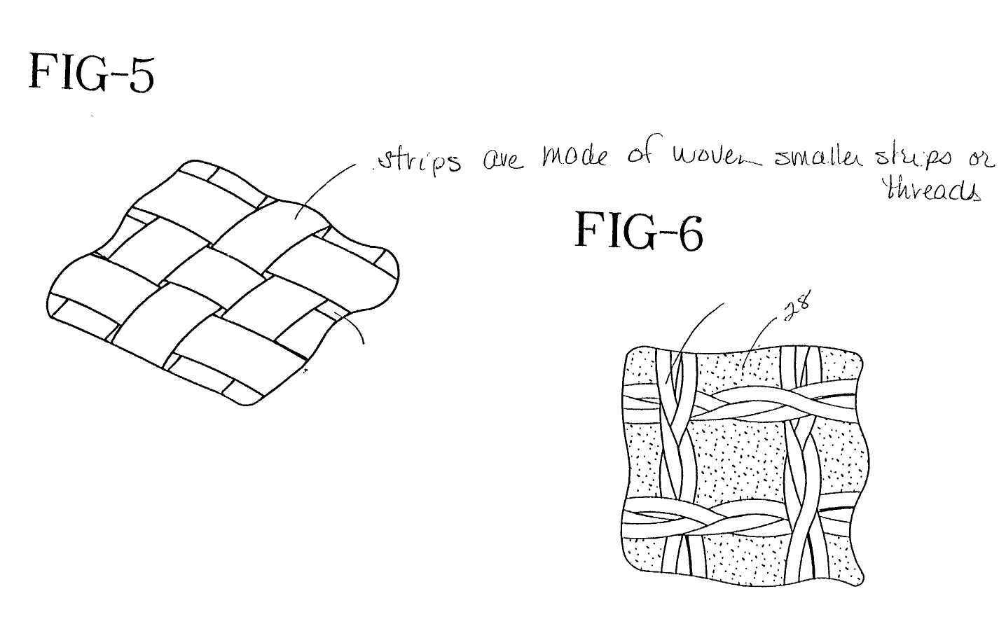

FIG. 4, depicts a desired embodiment of the present invention in which incorporates a continuous tubular inner body 7. This embodiment employs a non-continuous tubular body 2 of opposed wound or interwoven PTFE components. The woven or braided configuration may be two dimensional or may be three dimensional, as shown in FIGS. 5 and 6.

FIG. 5 shows two PTFE components, such as PTFE strips, or pre-manufactured PTFE tapes combined in a two dimensional matrix, wherein the tapes comprise the separate components of the non-continuous tubular body 2. The e.g. tapes may be interwoven closely, as shown in FIG. 5. In addition, closely woven tapes, filaments or strips may be used to form larger strips which may be used as components of the non-continuous tubular body.

FIG. 6 shows an enlarged view of a three dimensional thread comprised of three PTFE filaments braided together to form a three dimensional threads which may form the components of non-continuous tubular body. Such braided knitted or woven construction provides axial and radial compliance to the prosthesis by defining spaces within the braided, knitted or woven or extruded structure.

In certain applications where enhanced sealing properties are required, a sealant 28, as shown in FIG. 6, may be interspersed within the woven or braided components to create a non-porous tubular body. Sealants which may be used in the prosthesis include FEP, polyurethane, and silicone. Additional sealants include biological materials such as collagen, and hydrogels, polymethylmethacrylate, polyamide, and polycarbonate. Elastomers as sealants will have less impact on flexibility. A suitable sealant provides a substantially sealed outer tube without significantly reducing longitudinal and axial compliance.

As shown herein the braided longitudinally non-continuous tubular body shown in the above-referenced figures form non-continuous bodies comprised of PTFE components tubularly assembled. The non-continuous structure of the braided tubular body provides the composite prosthesis with enhanced radial and longitudinal, or axial compliance. The radial and axial compliance can, in fact, be varied with the different non-continuous PTFE bodies which may be used, as may be suitable for the use of the intraluminal prosthesis. The non-continuous layer 2 is formed by wrapping one, two, or three, or more PTFE tapes about, or through, each other.

In preferred embodiments the PTFE components are pre-manufactured tape of expanded PTFE (ePTFE), The term expanded refers to PTFE which has been stretched uniaxially, biaxially, or multiaxially in a particular direction. The PTFE tape of the prosthesis of the present invention is typically stretched in the longitudinal direction of the tape. When two or more tapes are combined to form the braided body, the resultant tubular body possesses a biaxial, or multiaxial resultant orientation in the aggregate. Because ePTFE exhibits increased strength in the direction of its stretching, the ePTFE tubularly assembled body exhibits the advantage of the increased strength of a biaxial or multiaxial stretched film, but exhibits the advantages of compliance because of its non-continuous surface. In another embodiment, PTFE filaments may be wound about a mandrel or support structure to form the non-continuous tubular body.

The continuous PTFE tubular layer may be bonded to the non-continuous PTFE tubular layer through spaces in the open wall of the stent. The bonding may be effectuated with the use of an adhesive, or by adhering the layers together without an adhesive. Bonding of the PTFE layers without an adhesive may take place by such methods as laminating, or sintering of the prosthesis. Furthermore, the stent may be adhered to the continuous PTFE tubular layer, the braided PTFE tubular layer, or both. Similarly, such adherence may take place with or without the use of an adhesive.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. An implantable composite intraluminal prosthesis comprising,
a circumferentially distensible tubular support structure having opposed inner and outer surfaces; and
a longitudinally; non-continuous tubular body on at least one surface of the support structure, comprising PTFE components, each having a length and a width, said length being greater than said width; said components being spirally wound, and positioned on said at least one surface.

2. A composite intraluminal prosthesis according to claim 1 wherein the longitudinally non-continuous polytetrafluoroethylene tubular body comprises at least two opposed spirally wound polytetrafluoroethylene components.

3. A composite intraluminal prosthesis as in claim 2 wherein the opposed polytetrafluoroethylene components are interwoven.

4. A composite intraluminal prosthesis as in claim 3 wherein said PTFE components comprise woven PTFE threads.

5. A composite intraluminal prosthesis as in claim 4 wherein at least one of said polytetrafluoroethylene threads is a braided thread comprising at least three PTFE filaments.

6. A composite intraluminal prosthesis according to claim 5 wherein a sealant is interspersed between said filaments.

7. A composite intraluminal prosthesis as in claim 1, further comprising,
another longitudinally, non-continuous PTFE tubular body on the other surface of the distensible support structure, said another non-continuous tubular body, comprising spirally wound PTFE components having a length and a width, said length being greater than said width; said another tubular body being secured to said longitudinally non-continuous tubular body.

8. The composite intraluminal prosthesis of claim 1, further comprising:
a continuous tubular ePTFE tubular body on the other surface of the distensible support structure, said continuous tubular body comprising spirally wound PTFE components having a length and a width, said length being greater than said width; said continuous tubular body secured to said longitudinally non-continuous tubular body to form said prosthesis.

9. A composite intraluminal prosthesis according to claim 8 wherein the longitudinally non-continuous polytetrafluoroethylene tubular body comprises at least two opposed spirally wound polytetrafluoroethylene components.

10. A composite intraluminal prosthesis as in claim 9 wherein the opposed polytetrafluoroethylene components are interwoven.

11. A composite intraluminal prosthesis as in claim 10 wherein said PTFE components comprise woven PTFE threads.

12. A composite intraluminal prosthesis as in claim 11 wherein at least one of said polytetrafluoroethylene threads is a braided thread comprising at least three PTFE filaments.

13. A composite intraluminal prosthesis according to claim 12 wherein a sealant is interspersed between said filaments.

14. A method of making an implantable composite intraluminal prosthesis, comprising:
a) providing a circumferentially distensible tubular support structure having opposed inner and outer surfaces;
b) providing at least two ePTFE components, each having a length greater than its width; and,
c) spirally winding the components to form a non-continuous tubular body on at least one surface of the tubular support structure to make a composite with a support structure side and an ePTFE side.

15. The method of claim 14, further comprising:
a) providing a continuous tubular body on the other surface of the distensible support structure, and
b) securing the continuous tubular body to the discontinuous tubular body.

16. The method of claim 14, further comprising:
spirally winding at least two PTFE components to form another longitudinally non-continuous tubular layer on the other surface of the distensible support structure.

17. A method according to claim 14 wherein at least one of the PTFE components comprises a PTFE thread, comprising two or more polytetrafluoroethylene filaments arranged in a braided configuration.

18. A method according to claim 14 wherein the PTFE components are interwoven.

19. A method according to claim 14 wherein at least two of the PTFE strips are wound in opposed spirals.

20. A method according to claim 17 wherein a sealant is interspersed between said filaments.
